(19) United States
(12) Patent Application Publication
(10) Pub. No.: US 2018/0078416 A1
(43) Pub. Date: Mar. 22, 2018

(54) INLET TUBE PROTECTOR FOR GLAUCOMA SHUNTS

(71) Applicant: Gregory Christiansen, Salt Lake City, UT (US)

(72) Inventor: Gregory Christiansen, Salt Lake City, UT (US)

(21) Appl. No.: 15/710,822

(22) Filed: Sep. 20, 2017

Related U.S. Application Data
(60) Provisional application No. 62/397,764, filed on Sep. 21, 2016.

(51) Int. Cl. A61F 9/007

(52) U.S. Cl. CPC A61F 9/00781; A61F 2250/0013; A61F 2220/0008; A61F 2230/001; A61F 2250/0039

(57) ABSTRACT

An inlet tube cover for a glaucoma shunt is provided. The glaucoma shunt may include an elongate inlet tube having a lumen, a first end, and a second end. The first end of the elongate tube is inserted into the eye to place the lumen in contact with aqueous humor and a reservoir which is attached to the second end of the inlet tube. The reservoir body may have a valve which is in fluid communication with the inlet tube lumen and which opens at a predetermined pressure to allow flow through the lumen of the inlet tube. The reservoir body is attached to the eye. The inlet tube cover is formed from a flexible polymer and includes a channel formed along a length thereof on a bottom surface thereof. The cover is attached to the eye with the inlet tube disposed in the channel so that the cover protects the inlet tube and assists in securing the inlet tube to the eye.

### RELATED APPLICATIONS

The present application claims the benefit of U.S. Provisional Application Ser. No. 62/397,764, filed Sep. 21, 2016, which is expressly incorporated herein by reference in its entirety.

### THE FIELD OF THE INVENTION

The present invention relates to glaucoma shunts. In particular, examples of the present invention relates to a system for protecting the drainage (inlet) tube of a glaucoma shunt.

### BACKGROUND

Glaucoma is a group of eye diseases which result in damage to the optic nerve and vision loss. Glaucoma patients may suffer from increased intraocular pressure and this increased pressure may increase the risk of vision loss. Glaucoma is treated to avoid damage to the eye and to preserve vision. Glaucoma is often initially treated with medication aimed at reducing intraocular pressure or with treatments such as laser therapy aimed at increasing the outflow of aqueous humor. In cases where other therapy does not resolve this, a glaucoma shunt may be used to decrease intraocular pressure by providing a new pathway for the outflow of aqueous humor.

### BRIEF DESCRIPTION OF THE DRAWINGS

Non-limiting and non-exhaustive examples of the present invention are described with reference to the following figures, wherein like reference numerals refer to like parts throughout the various views unless otherwise specified.

FIG. 1 is a drawing which shows a perspective view of an eye, glaucoma shunt, and inlet tube cover.

FIG. 2 is a drawing which shows a cross-sectional view of the eye, glaucoma shunt, and inlet tube cover.

FIG. 3 is a drawing which shows a top view of the inlet tube cover.

FIG. 4A is a drawing which shows a bottom view of the inlet tube cover.

FIG. 4B is a drawing which shows a bottom view of the inlet tube cover.

FIG. 5 is a drawing which shows a side view of the inlet tube cover.

FIG. 6 is a drawing which shows a front view of the inlet tube cover.

FIG. 7 is a drawing which shows a back view of the inlet tube cover.

FIG. 8 is a drawing which shows a front view of the inlet tube cover, inlet tube, and eye.

FIG. 9 is a top view drawing of the inlet tube cover.

FIG. 10A is a bottom view drawing of the inlet tube cover of FIG. 9.

FIG. 10B is an alternate bottom view drawing of the inlet tube cover of FIG. 9.

FIG. 11 is a right side drawing of the inlet tube cover of FIG. 9.

FIG. 12 is a left side drawing of the inlet tube cover of FIG. 9.

FIG. 13 is a front view drawing of the inlet tube cover of FIG. 9.

FIG. 14A is a back view drawing of the inlet tube cover of FIG. 9 with the lower surface shown in FIG. 10A.

FIG. 14B is a back view drawing of the inlet tube cover of FIG. 9 with the lower surface shown in FIG. 10B.

FIG. 15 is a perspective drawing of the inlet tube cover of FIG. 9.

FIG. 16 is a top view drawing of an alternate embodiment of the inlet tube cover.

FIG. 17 is a bottom view drawing of the inlet tube cover of FIG. 16.

FIG. 18 is a right side drawing of the inlet tube cover of FIG. 16.

FIG. 19 is a left side drawing of the inlet tube cover of FIG. 16.

FIG. 20 is a front view drawing of the inlet tube cover of FIG. 16.

FIG. 21 is a back view drawing of the inlet tube cover of FIG. 16.

FIG. 22 is a perspective drawing of the inlet tube cover of FIG. 16.

FIG. 23 is a top view drawing of an alternate embodiment of the inlet tube cover.

FIG. 24 is a bottom view drawing of the inlet tube cover of FIG. 23.

FIG. 25 is a right side drawing of the inlet tube cover of FIG. 23.

FIG. 26 is a left side drawing of the inlet tube cover of FIG. 23.

FIG. 27 is a front view drawing of the inlet tube cover of FIG. 23.

FIG. 28 is a back view drawing of the inlet tube cover of FIG. 23.

FIG. 29 is a perspective drawing of the inlet tube cover of FIG. 23.

Corresponding reference characters indicate corresponding components throughout the several views of the drawings. Skilled artisans will appreciate that elements in the figures are illustrated for simplicity and clarity. Unless noted, the drawings have been drawn to scale to increase understanding of the invention. Also, common but well-understood elements that are useful or necessary in a commercially feasible embodiment may not be depicted in order to facilitate a less obstructed view of these various embodiments of the present invention.

It will be appreciated that the drawings are illustrative and not limiting of the scope of the invention which is defined by the appended claims. The examples shown each accomplish various different advantages. It is appreciated that it is not possible to clearly show each element or advantage in a single figure, and as such, multiple figures are presented to separately illustrate the various details of the examples in greater clarity. Similarly, not every example need accomplish all advantages of the present disclosure.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

In the above disclosure, reference has been made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be used in connection with other embodiments whether or not explicitly described. The particular features, structures or characteristics may be combined in any suitable combination and/or sub-combinations in one or more embodiments or examples. It is appreciated that the figures provided herewith are for explanation purposes to persons ordinarily skilled in the art. The figures are drawn to scale to increase understanding of the invention.

The disclosure describes a tube protector for glaucoma shunts. Particularly, the present disclosure describes a manufactured tube protecting cover which may be surgically installed onto a patient's eye during surgical installation of a glaucoma shunt to thereby secure the glaucoma shunt tube to the patient's eye. The tube protector covers a section of the glaucoma shunt tube and protects the glaucoma shunt tube from movement and erosion which may occur during movement of the patient's eye.

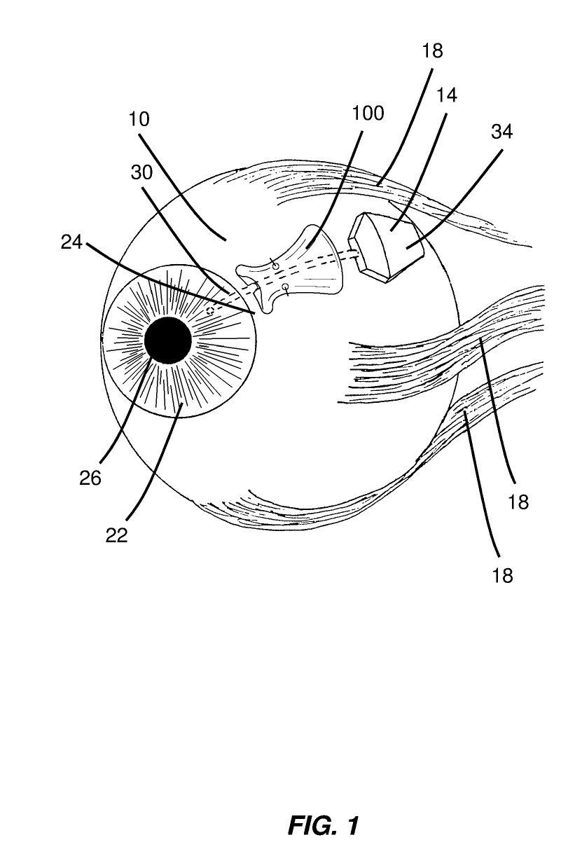

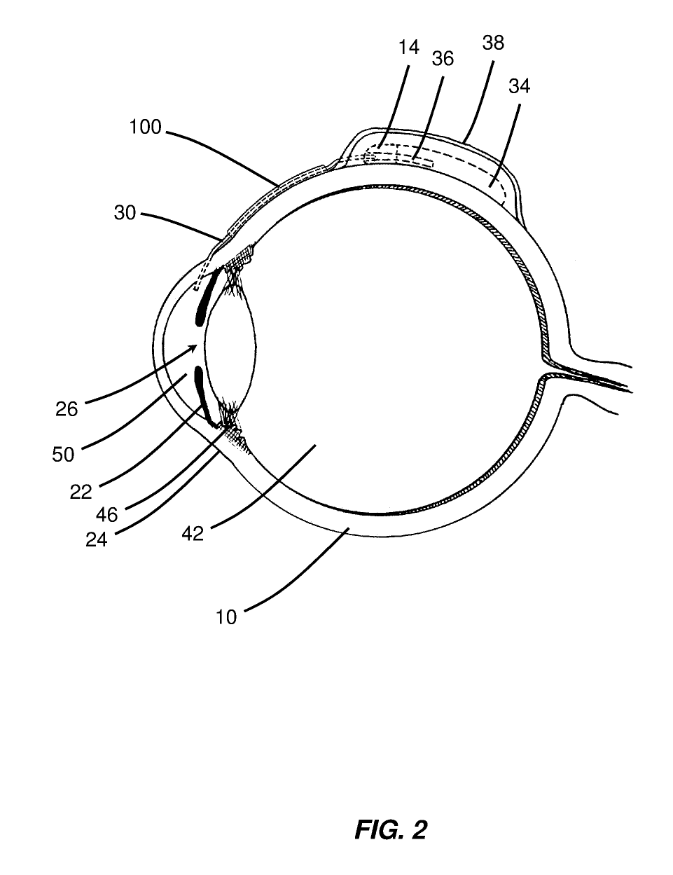

Turning now to FIG. 1, a drawing of an eye 10, glaucoma shunt 14 (e.g. a seton), and tube protection cover 100 is shown. The eye 10 is shown apart from many additional anatomical structures to facilitate illustration of the glaucoma shunt and cover. It is understood that the glaucoma shunt and cover are used on the eye of a living patient to treat the patient. The eye 10 is illustrated with the eye muscles 18, iris 22, and pupil 26. The glaucoma shunt 14 includes an inlet tube 30 and a reservoir plate 34. The inlet tube 30 is often a silicone tube with an outside diameter of between about 0.5 mm and about 1 mm. and lumen which is between about 0.25 mm and 0.7 mm in diameter. The reservoir plate 34 is often made with an outer case (which may have two or more pieces attached together) and may have an inner membrane. The reservoir plate 34 forms a reservoir which receives aqueous humor from the eye. The reservoir plate 34 may include a valve 36 (FIG. 2), such as which may be formed between adjoining lips of a folded internal membrane, which opens at a predetermined pressure and allows liquid to flow from the eye, through the inlet tube 30, through the valve 36, and into the reservoir formed by the reservoir plate 34. The liquid then flow out of the reservoir plate 34. Thus, in a simple configuration, the glaucoma shunt 14 may include an inlet tube 30 connected to a reservoir 34, and the reservoir 34 may include a membrane/valve 36 which is attached to the inlet tube 30 and which allows liquid to flow from the inlet tube 30 to into the reservoir 34 at a desired opening pressure such as 10 mm of Hg to maintain a desired intraocular pressure in the eye 10.

The glaucoma shunt 14 is implanted in the eye by inserting the inlet tube 30 into the intraocular chamber of the eye 10. The inlet tube 30 is inserted adjacent the iris 22. The inlet tube 30 is typically inserted into the eye through the eye sclera at a point near the limbus 24. Once the inlet tube 30 is inserted into the eye 10, the reservoir plate 34 is attached to the eye. The reservoir plate 34 is typically attached to the eye at a point between the eye muscles 18, such as on a side of the eye at a point approximately 9 mm to 10 mm posterior to the limbus 24. The reservoir plate 34 is often held to the eye by sutures.

Attachment of the reservoir plate 34 to the side of the eye 10 as shown leaves a length of the inlet tube 30 exposed along the outside of the eye. The cover 100 is attached to the exterior of the eye 10 to cover the inlet tube 30 between the insertion point near the limbus 24 and the reservoir plate 34. The cover 100 may not always completely cover the exposed portion of the inlet tube 30, but covers a majority of the inlet tube 30 from a location adjacent the point of insertion into the eye to a location adjacent the reservoir plate 34. The cover 100 covers the inlet tube 30 and protects the inlet tube 30 and eye 10 from damage such as erosion through the conjunctiva. If a tube erodes through the conjunctiva, the eye is at risk for sight threatening infections which can cause irreversible blindness.

After installation, aqueous humor flows from the eye 10 through the inlet tube 30, and into the reservoir plate 34. After the glaucoma shunt 14 is implanted onto an eye, a bleb is typically formed around the reservoir plate 34. The bleb is a tissue membrane which surrounds the shunt body and aqueous humor which enters the reservoir plate 34 from the inlet tube 30 is absorbed into vascular tissues of the patient through the bleb.

FIG. 2 shows a cross-sectional view of the eye 10 with the implanted glaucoma shunt 14 and tube cover 100. It can be seen how the inlet tube 30 may be inserted into the eye adjacent the limbus 24 and extend to a position near the iris 22. The inlet tube 30 may be clear silicone and, being quite thin, is not easily seen. FIG. 2 illustrates how a bleb 38 may be formed around the reservoir plate 34. The inlet tube cover 100 covers a majority of the length of the inlet tube 30 between the point of insertion near the limbus 24 and the reservoir plate 34. Aqueous humor flows from the ciliary body 46 into the anterior chamber 50, into the inlet tube 30, and to the reservoir plate 34 to maintain the intraocular pressure at a desired level.

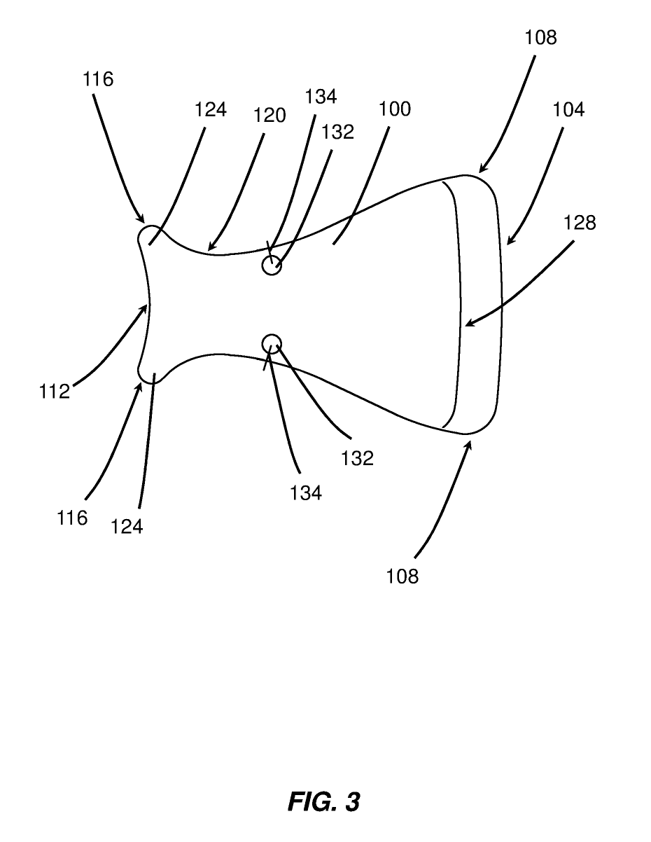

FIGS. 3 through 7 show top, bottom, side, front, and back views of the inlet tube cover 100, respectively. The inlet tube cover 100 is formed from a flexible material and, in a preferred configuration, is formed from a flexible polymer material such as medical grade silicone. As illustrated in FIG. 3, the inlet tube cover 100 has a general hourglass shape when viewed from the top. The inlet tube cover 100 is approximately 12 mm long from the front to the back. The back 104 of the inlet tube cover 100 is approximately 10 mm wide and the back edge may be gently curved forwards, forming a convex edge, and may transition into rounded corners 108 which have radii of between about 1 mm to about 2 mm. The front 112 of the inlet tube cover 100 is approximately 6 mm wide and the front edge may be rounded and define an edge which is concave with a radius of approximately 15 mm. The front edge of the inlet tube cover 100 may transition into rounded corners 116 which have radii of between about 0.5 mm and about 1 mm.

The sides of the inlet tube cover 100 are curved and are concave in shape. These sides form a narrowed waist 120 between the front 112 and the back 104 of the inlet tube cover 100. The narrowed waist 120 of the hourglass shape may be about 4 mm wide. The narrowest part of the narrowed waist 120 may be located between about 2 mm and about 5 mm from the front of the inlet tube cover 100, making the front portion of the hourglass shape typically shorter and narrower than the rear portion of the hourglass shape. The front corners 116 transition into the concave sides and the narrowed waist 120; forming front wings 124 which are between about 1 mm and about 2 mm long and between about 0.5 mm and about 1.5 mm wide. The back corners 108 transition into the waist 120, forming a generally triangular back portion.

A cut line 128 may be located between about 1 mm and about 2 mm from the rear edge 104 of the inlet tube cover 100. The cut line 128 extends generally parallel to the rear edge 104 and provides a guide to a surgeon to allow the surgeon to easily reduce the overall size of the inlet tube cover 100. If desired, the cut line may be omitted and multiple sizes of the inlet tube cover 100 may be provided as desired. The cut line 128 may be molded or otherwise formed into the underside of the inlet tube cover 100 in order to provide a smooth upper surface.

Attachment points 132 may be formed in the inlet tube cover 100. The attachment points 132 may be formed as holes through the inlet tube cover 100. Alternatively, the attachment points 132 may be formed as depressions in the top surface of the inlet tube cover 100, and may be between about 0.5 mm and about 1 mm in diameter and between about 3 mm and about 5 mm, or about 4 mm back from the front of the inlet tube cover 100. Although the inlet tube cover 100 is relatively thin at its edges, the attachment points 132 may be formed so that they are thinner than the surrounding material. The attachment points 132 may be formed so that the material in the attachment point has a thickness which is between about 0.1 mm and 0.5 mm. The attachment points 132 provide a point of reduced thickness and easy visual identification for suturing the inlet tube cover 100 to the eye with sutures 134. The reduced thickness of the inlet tube cover 100 at the attachment points 132 provides for easier suturing of the inlet tube cover 100 to the eye 10. In some cases, the inlet tube cover 100 need not be sutured to the eye 10. In these cases, the attachment points 132 do not interfere with the use of the inlet tube cover 100. The inlet tube cover 100 may remain continuous across the attachment points 132 as the attachment points 132 need not perforate the inlet tube cover 100. The top, outside, surface of the inlet tube cover 100 may be smooth and have rounded edges.

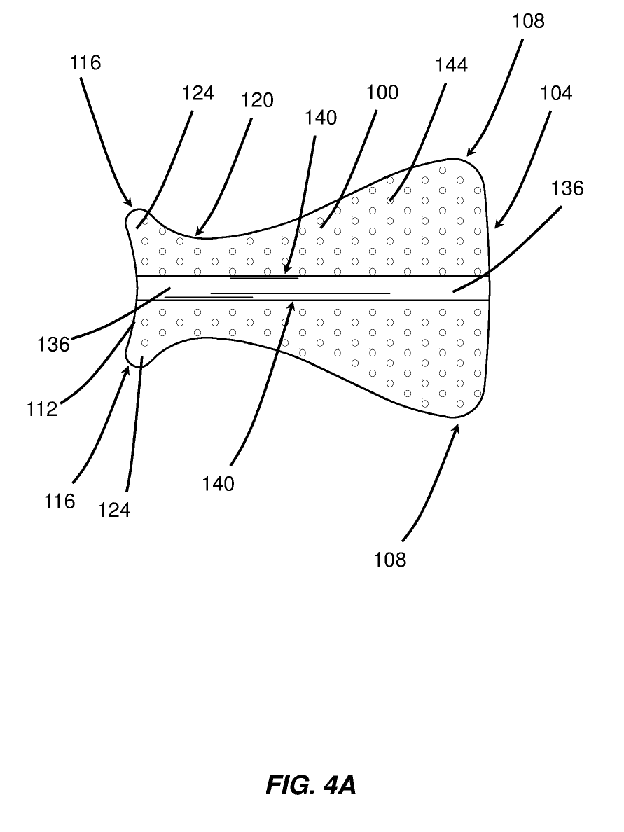

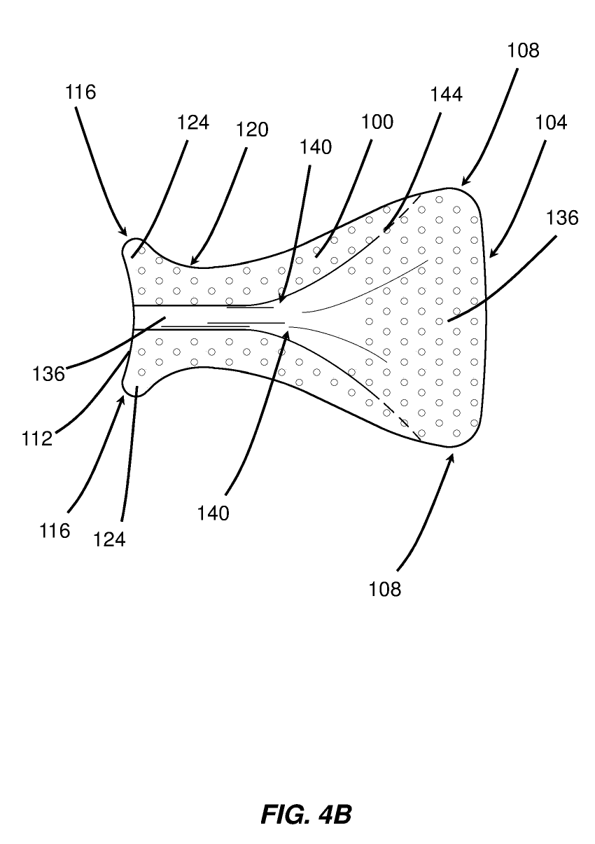

FIG. 4A shows a bottom view of the inlet tube cover 100. FIG. 4A shows how the inlet tube cover 100 may include a channel 136 formed along the length of the bottom surface of the inlet tube cover 100. The channel 136 extends from the front edge 112 to the rear edge 104 of the inlet tube cover 100 and extends through the front and rear faces of the inlet tube cover 100. The channel 136 receives the inlet tube 30 and holds the inlet tube 30 between the eye 10 and the inlet tube cover 100. The channel 136 is typically between about 0.5 mm and about 1 mm in width and depth so that the inlet tube 30 is easily received in the channel 136. The channel 136 may have lengthwise bottom edges 140 which extend along the length of the channel. In one embodiment, the bottom edges 140 may have a distance between the two bottom edges 140 which is slightly less than the diameter of the inlet tube 30 to retain the inlet tube 30 in the channel 136.

During installation, the inlet tube cover 100 may be bent slightly to open the channel 136 and receive the inlet tube 30 and the inlet tube cover 100 may then be relaxed to capture the inlet tube 30 in the channel 136. In another embodiment, the channel 136 may have bottom edges 140 with a distance between the bottom edges which is slightly greater than the diameter of the inlet tube 30 to make it easier to place the inlet tube 30 in the channel 136.

The bottom surface of the inlet tube cover 100 may have small dimples or stippling, indicated generally at 144 as circles, to allow surface tension to hold the inlet tube cover 100 against the outside surface of the eye 10. These dimples or stippling may be molded into the bottom surface of the inlet tube cover 100. The dimples 144 are typically quite small and may be between about 0.2 mm and about 0.5 mm in diameter and between about 0.1 mm and about 0.3 mm in depth.

FIG. 4B shows an alternate bottom view of the inlet tube cover 100. As illustrated, the channel 136 may be formed along the length of the inlet tube cover 100 and may, after traversing a distance from the front edge 112 towards the rear edge 104 of the inlet tube cover 100, gradually widen and transition into a smooth lower surface at the rear of the inlet tube cover 100. As shown, the channel 136 extends between approximately 3 mm and approximately 5 mm, or by approximately 4 mm from the front edge 112 and then widens towards the rear edge 104. The channel 136 transitions into a smooth and open area so that the rear portion of the inlet tube cover 100 is generally flat on its bottom surface (apart from dimples or stippling 144 if used) and is generally thin and of uniform thickness at its rear edge 104. The channel 136 receives the inlet tube 30 and holds the inlet tube 30 between the eye 10 and the inlet tube cover 100. Except for the modification shown, the channel 136 may otherwise include the structures and functions described above. Particularly, the side edges 140 of the channel 136 near the front of the channel may be slightly narrower than the width of the inlet tube 30 to capture the inlet tube 30. Alternatively, the side edges 140 of the front portion of the channel 136 may be slightly wider than the width of the inlet tube 30 and may be rounded to allow for easier insertion of the inlet tube 30.

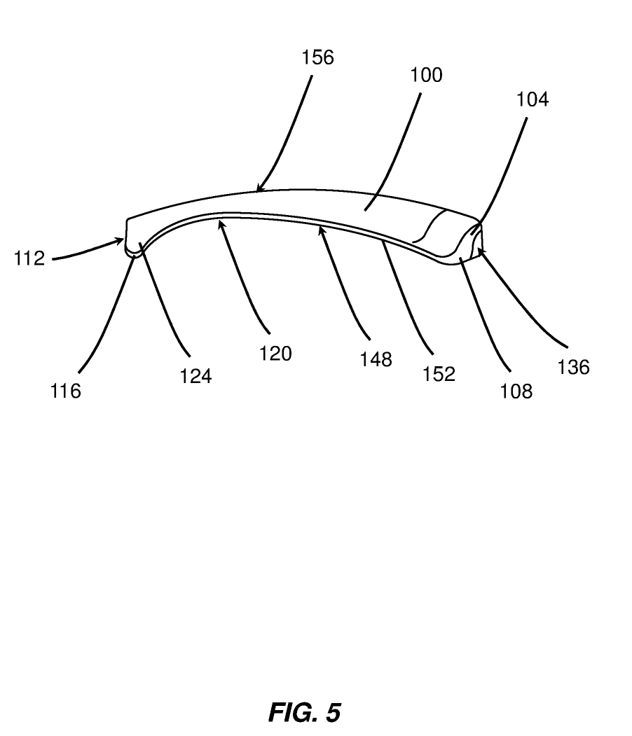

FIG. 5 shows a side view of the inlet tube cover 100 and illustrates how the inlet tube cover 100 is curved as viewed from the side (from the front to the back). The inlet tube cover 100 is curved from the front to the back and also from the side to the side, having a generally spherical lower surface 148. The inlet tube cover 100 is curved so that the bottom (inside) surface 148 of the inlet tube cover 100 has a radius of about 12 mm. The upper surface 156 of the inlet tube cover 100 is also curved, and has a radius of curvature from front to back which is about 12 mm. This provides a fairly uniform thickness of the inlet tube protector 100 along its length. The side edges 152 of the inlet tube cover 100 are thin, and typically have a thickness between about 0.1 mm and 0.5 mm. The upper edge of the side edges 152 (the upper perimeter corner of the inlet tube cover 100) is typically rounded or beveled to create a smooth upper surface.

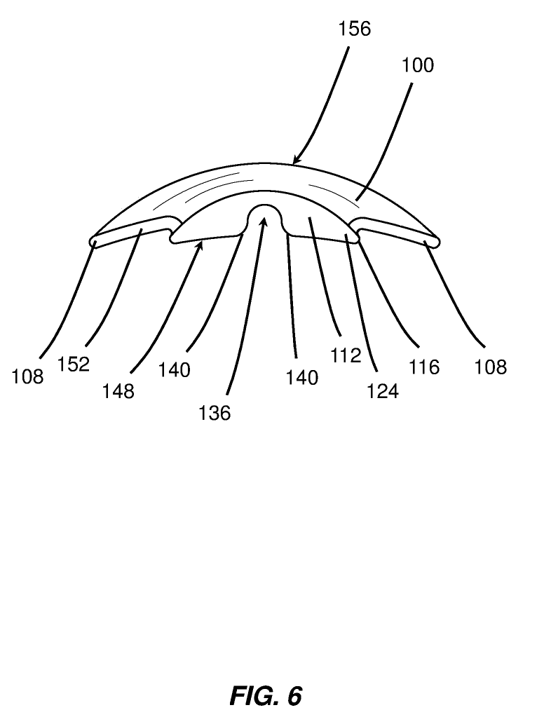

FIG. 6 shows a front view of the inlet tube cover 100. It can be seen how the bottom surface 148 of the inlet tube cover 100 is also curved with a radius of about 12 mm as viewed from the front (curving side to side). Thus, the inlet tube cover 100 has a lower surface 148 which is generally spherical with a radius of about 12 mm. It can be seen how the center of the inlet tube cover 100 in the front to back direction is thicker than the sides of the inlet tube cover 100 to accommodate the inlet tube 30. Accordingly, the upper surface 156 of the inlet tube cover 100 has a radius of curvature in the side to side direction which is less than the radius of curvature of the lower surface 148 of the inlet tube cover 100. The side to side radius of curvature of the front portion of the upper surface 156 of the inlet tube cover may be between approximately 7 mm and approximately 10 mm. This may be an average curvature of the upper surface 156, as the upper surface 156 may curve more above the channel 136 and inlet tube 30 and may curve less along the sides of the inlet tube cover 100.

The inlet tube cover 100 is typically between about 1 mm and about 1.5 mm in thickness along the front to back center of the inlet tube cover 100. Of this thickness, the channel 136 is typically between about 0.5 mm and 1 mm and there is about 0.5 mm thick section of material across the top of the channel 136. The upper surface 156 of the inlet tube cover 100 is contoured to allow for the center line thickness along the channel 136 and transitions to the side edges 152 which are thinner. It can be seen how the front edge 112 is taller near the center and shorter near the side edges 152. It can be seen how the edges of the inlet tube cover 100 may be rounded to avoid sharp corners. In one example, the upper surface edges of the inlet tube cover 100 may be rounded or beveled while the lower surface edges of the inlet tube cover 100 may be left square to stabilize the inlet tube cover 100 on the eye from fluid tension. In another example, both the upper and lower surface edges of the inlet tube cover may be rounded or beveled to ensure a smooth device without sharp corners.

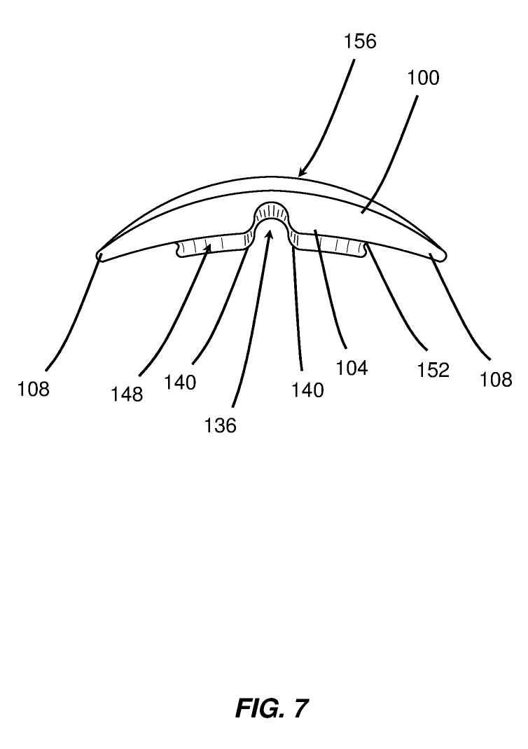

FIG. 7 shows a back view of the inlet tube cover 100. The back edge 104 of the inlet tube cover 100 has similar contour characteristics as the front edge 112, although it is wider than the front edge. It can be seen how the bottom surface 148 of the inlet tube cover 100 is curved with a radius of about 12 mm as viewed from the back (curving side to side). The inlet tube cover 100 has a lower surface 148 which is generally spherical with a radius of about 12 mm. It can be seen how the center of the inlet tube cover 100 in the front to back direction is thicker than the sides of the inlet tube cover 100 to accommodate the inlet tube 30. Accordingly, the upper surface 156 of the rear portion of the inlet tube cover 100 has a radius of curvature in the side to side direction which is less than the radius of curvature of the lower surface 148 of the inlet tube cover 100, and which may be approximately 10 mm.

The inlet tube cover 100 is typically between about 1 mm and about 1.5 mm in thickness at the center of the inlet tube cover 100, allowing for a channel 136 which is typically between about 0.5 mm and 1 mm and about 0.5 mm thick section of material across the top of the channel 136. It can be seen how the rear edge 104 is taller/thicker near the center and shorter/thinner near the side edges 152. It can be seen how the edges of the inlet tube cover 100 may be rounded to avoid sharp corners.

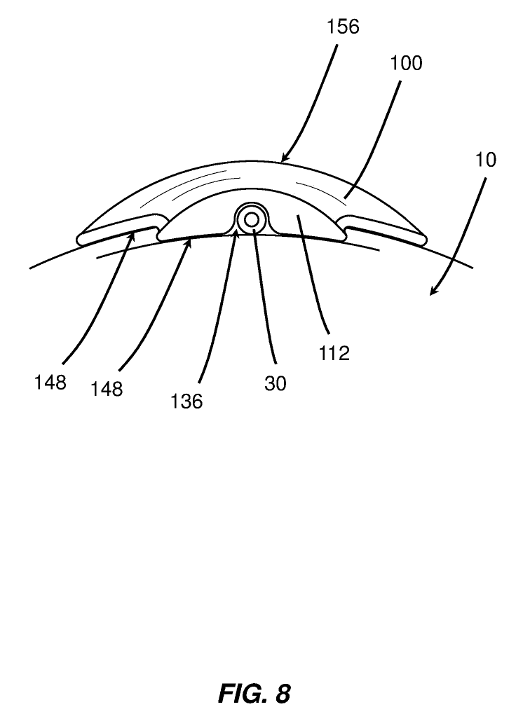

FIG. 8 shows a front view of the inlet tube cover 100 after the shunt 14 and the inlet tube cover 100 has been installed on an eye 10. The shunt 14 is implanted in the eye by inserting the inlet tube 30 into the intraocular chamber of the eye 10. The inlet tube 30 is inserted adjacent the iris 22 (not shown) at a point near the limbus 24. Relative to the drawing, the insertion point for the inlet tube 30 is in front of the inlet tube cover 100. The reservoir plate 34 (not shown) is attached to the eye behind the inlet tube cover 100 with sutures. A length of the inlet tube 30 is exposed along the outside of the eye between the reservoir plate 34 and the insertion point of the inlet tube 30. The cover 100 is attached to the exterior of the eye 10 to cover the inlet tube 30 between the insertion point near the limbus 24 and the reservoir plate 34.

It can be seen how the bottom surface 148 of the inlet tube cover 100 is attached to the surface of the eye 10. The inlet tube cover 100 conforms to the eye 10. The inlet tube cover 100 has been attached to the eye 10 with sutures (not visible behind the front surface 112 of the inlet tube cover 100). The inlet tube cover 100 is also held to the eye 10 by surface tension, which may be assisted by lower surface dimples. The inlet tube cover 100 has been installed such that the inlet tube 30 is disposed in the channel 136 and is held between the inlet tube cover 100 and the eye 10. The inlet tube cover 100 protects the inlet tube 30 from damage and prevents erosion of the inlet tube 30. The inlet tube cover 100 also protects the eye 10 from damage such as erosion through the conjunctiva. The inlet tube cover 100 also helps to keep the inlet tube 30 in a desired position and, by better fixing the inlet tube 30, stabilizes the reservoir plate 34. The inlet tube cover 100 simplifies the surgery to install the glaucoma shunt 14 and typically improves the success of the glaucoma shunt 14.

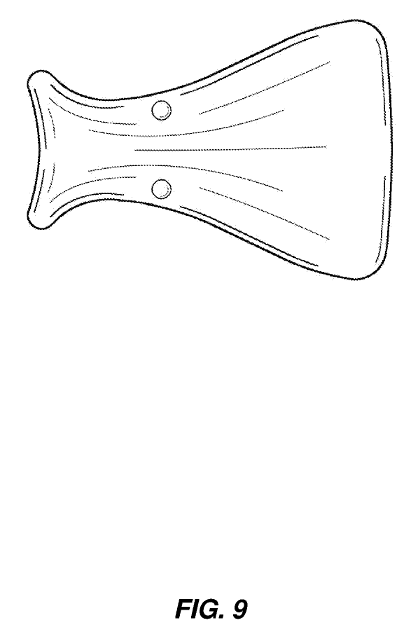

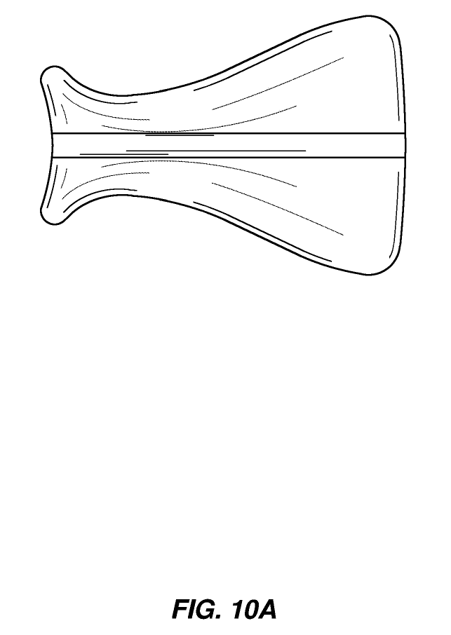

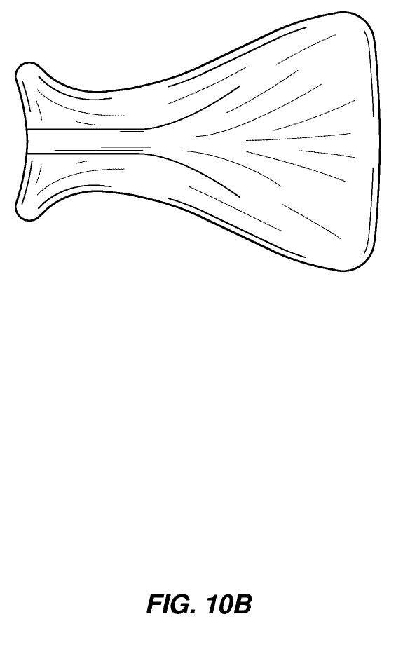

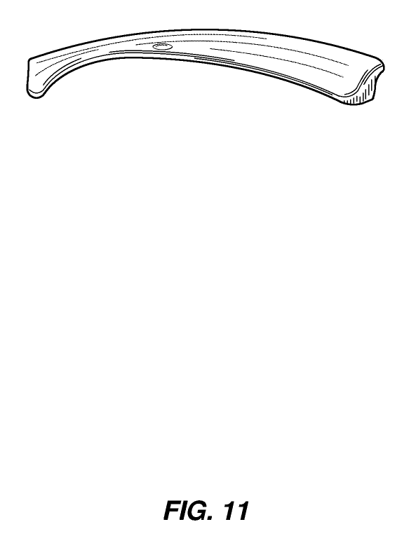

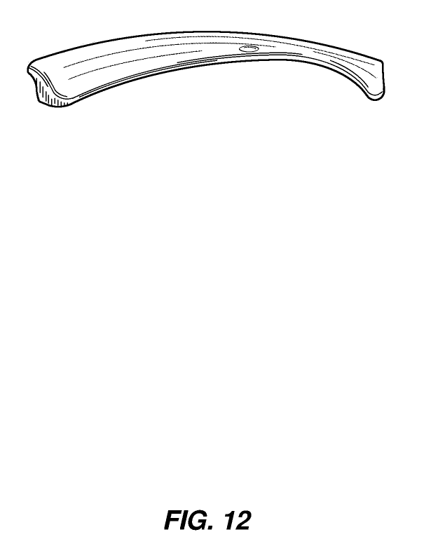

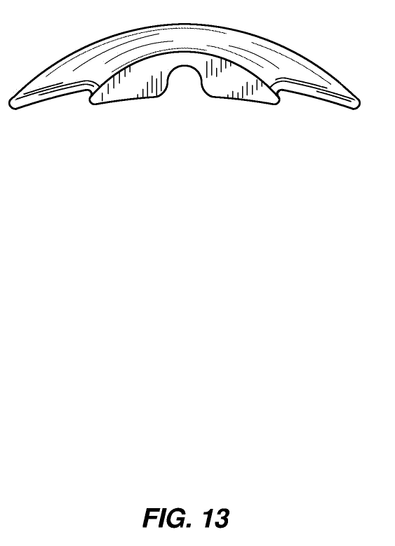

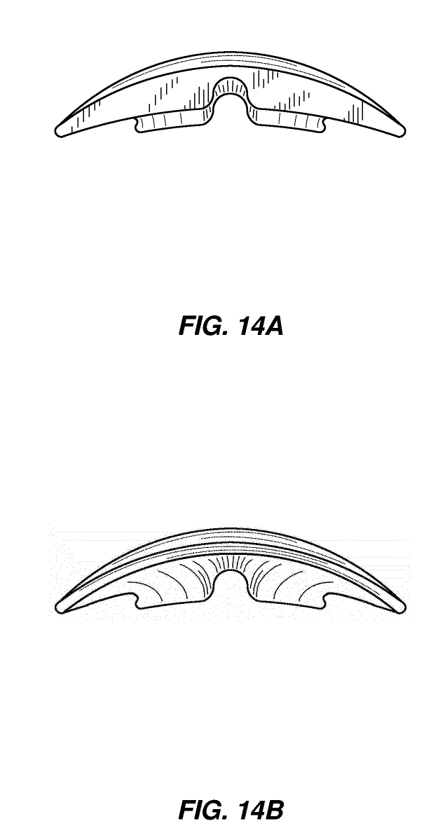

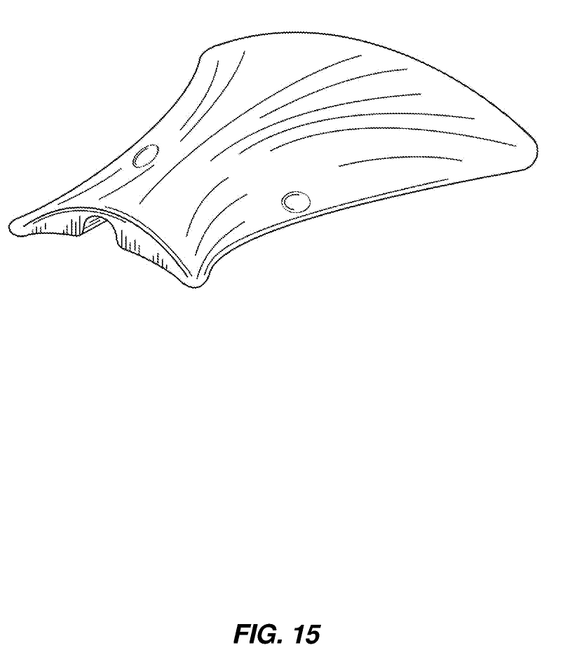

FIGS. 9 through 15 show shaded drawings of the inlet tube cover as disclosed above. The drawings are not numbered for clarity in showing the inlet tube cover, but include all of the elements and structures discussed above. FIG. 9 is a top view drawing of the inlet tube cover. FIG. 10A is a bottom view drawing of the inlet tube cover as shown in FIG. 4A. FIG. 10B is an alternate bottom view drawing of the inlet tube cover as shown in FIG. 4B. FIG. 11 is a right side drawing of the inlet tube cover. FIG. 12 is a left side drawing of the inlet tube cover. FIG. 13 is a front view drawing of the inlet tube cover. FIG. 14A is a back view drawing of the inlet tube cover with a channel as shown in FIG. 10A. FIG. 14B is a back view drawing of the inlet tube cover with a partial channel as shown in FIG. 10B. FIG. 15 is a perspective drawing of the inlet tube cover.

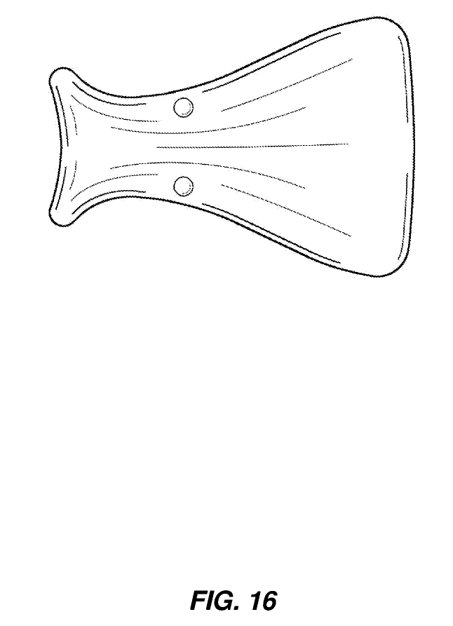

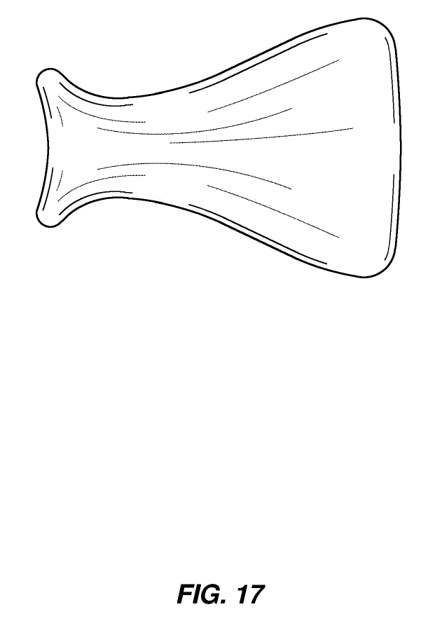

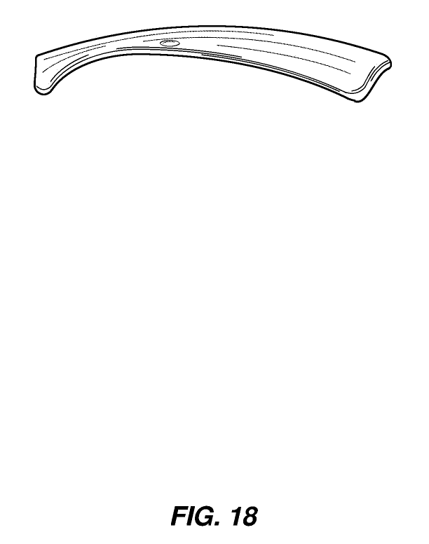

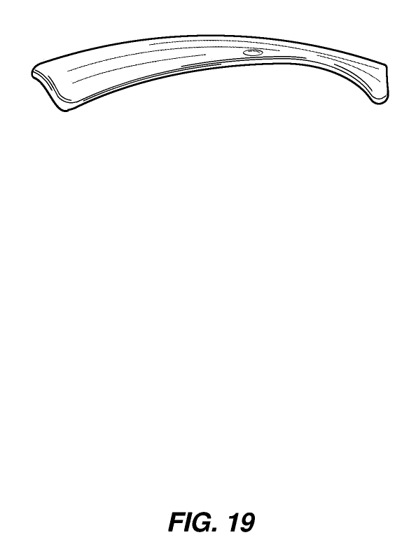

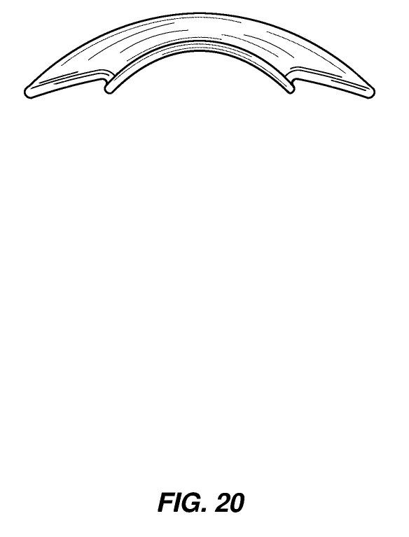

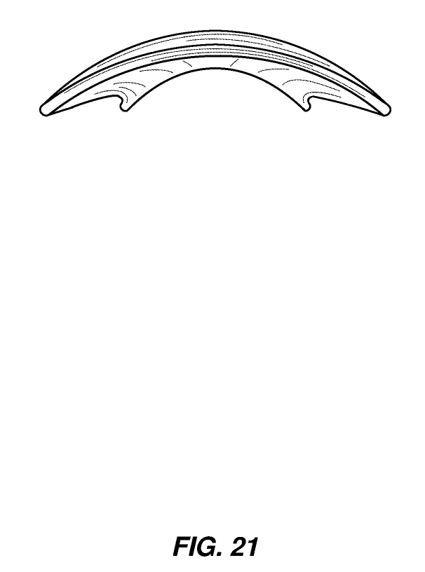

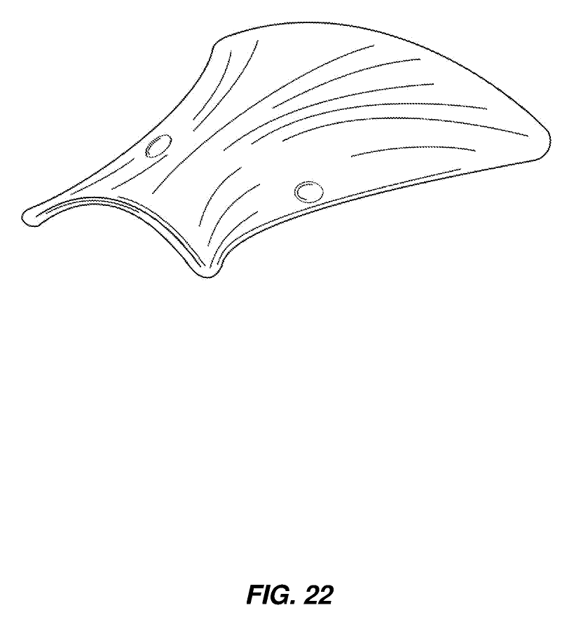

FIGS. 16 through 22 show an alternate embodiment of the inlet tube cover. The drawings are not numbered for clarity in showing the inlet tube cover, but include all of the elements and structures discussed above with respect to FIGS. 1 through 8 except as otherwise noted. Where the above inlet tube cover includes a channel in the bottom surface to receive the inlet tube, the inlet tube cover shown in FIGS. 16 through 22 does not include a narrow fitted channel, and instead includes a smoothly concave bottom surface which receives the inlet tube 30 and holds the inlet tube 30 between the inlet tube cover and the eye 10. FIG. 16 is a top view drawing of this embodiment of the inlet tube cover. FIG. 17 is a bottom view drawing of the inlet tube cover. FIG. 18 is a right side drawing of the inlet tube cover. FIG. 19 is a left side drawing of the inlet tube cover. FIG. 20 is a front view drawing of the inlet tube cover. FIG. 21 is a back view drawing of the inlet tube cover. FIG. 22 is a perspective drawing of the inlet tube cover.

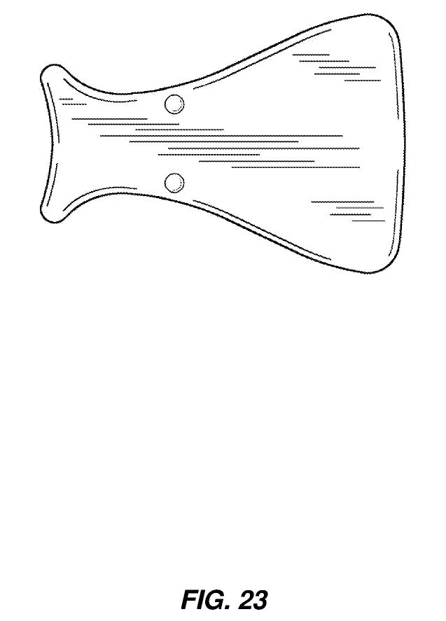

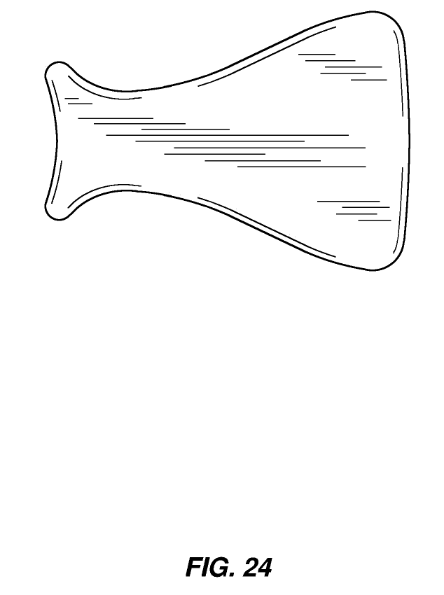

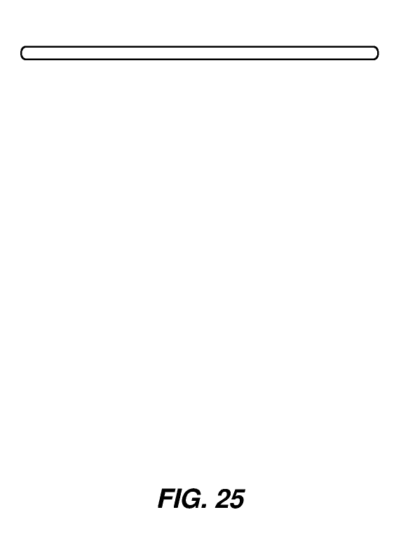

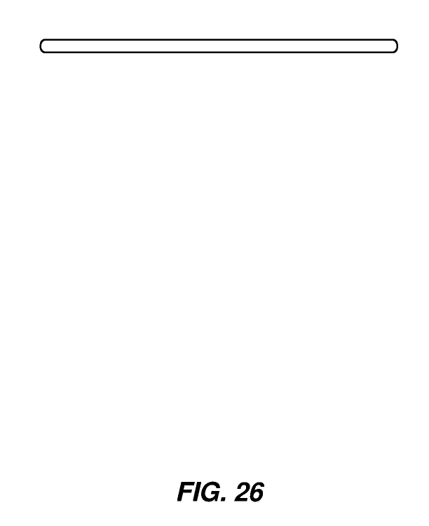

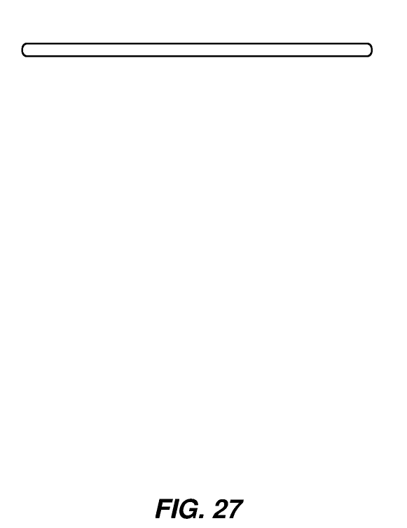

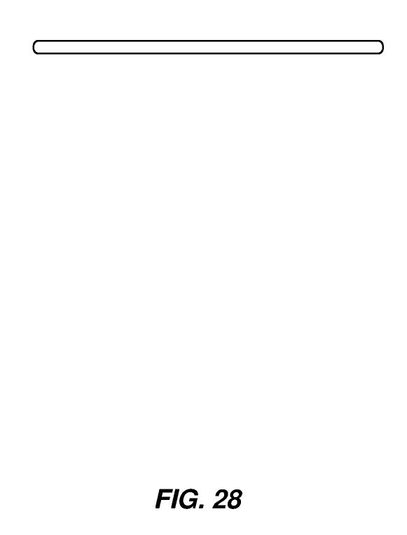

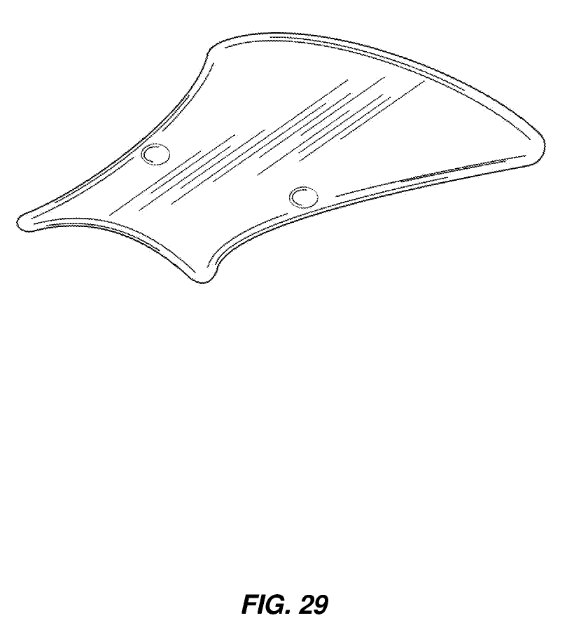

FIGS. 23 through 29 show an alternate embodiment of the inlet tube cover. The drawings are not numbered for clarity in showing the inlet tube cover, but include all of the elements and structures discussed above with respect to FIGS. 1 through 8 except as otherwise noted. The inlet tube cover shown in FIGS. 23 through 29 does not include a narrow fitted channel, and is instead formed from a flat piece of flexible and drape-able material. The inlet tube cover is attached to an eye in the same manner as is discussed above to secure the inlet tube between the inlet tube cover and the eye. As attached, the inlet tube cover forms into a generally spherical shape and the bottom surface will become generally concave against the eye. The upper surface of the inlet tube cover takes the general shape which is shown in FIGS. 1 and 15. FIG. 23 is a top view drawing of this alternate embodiment of the inlet tube cover. FIG. 24 is a bottom view drawing of the inlet tube cover of FIG. 23. FIG. 25 is a right side drawing of the inlet tube cover of FIG. 23. FIG. 26 is a left side drawing of the inlet tube cover of FIG. 23. FIG. 27 is a front view drawing of the inlet tube cover of FIG. 23. FIG. 28 is a back view drawing of the inlet tube cover of FIG. 23. FIG. 29 is a perspective drawing of the inlet tube cover of FIG. 23.

The embodiment of the inlet tube cover shown in FIGS. 16 through 22 and the embodiment of the inlet tube cover shown in FIGS. 23 through 29 are used and installed in the manner discussed with respect to FIGS. 1 through 8 above. Where the inlet tube cover includes a partial channel formed in the lower surface, the inlet tube is placed into the channel and the inlet tube cover is attached to the eye as discussed. Where the inlet tube cover has a smoothly concave lower surface without a channel, the inlet tube cover is placed over the inlet tube and onto the eye so that the inlet tube it located generally along the longitudinal center of the inlet tube cover and is located between the inlet tube cover and the eye. The inlet tube cover is then typically secured to the eye with sutures. The inlet tube cover takes the shape shown in the perspective view of FIG. 22 and is attached as shown in FIG. 1.

Where the inlet tube cover is formed from a thin, flat, and flexible material, the inlet tube cover has a smooth and flat upper and lower surface. The inlet tube cover is placed over the inlet tube and onto the eye so that the inlet tube it located generally along the longitudinal center of the inlet tube cover and is located between the inlet tube cover and the eye. The inlet tube cover is then typically secured to the eye with sutures. The inlet tube cover is attached as shown in FIG. 1. The inlet tube cover takes the shape shown in the perspective view of FIG. 22 when attached to the eye due to the flexibility and the drape-ability of the material used to make the inlet tube cover. Accordingly, the installed inlet tube cover takes the shape shown in FIGS. 1 and 22 and has a generally concave lower surface and a generally convex upper surface.

The above description of illustrated examples of the present invention, including what is described in the Abstract, are not intended to be exhaustive or to be limitation to the precise forms disclosed. While specific examples of the invention are described herein for illustrative purposes, various equivalent modifications are possible without departing from the broader scope of the present claims. Indeed, it is appreciated that specific example dimensions, materials, etc., are provided for explanation purposes and that other values may also be employed in other examples in accordance with the teachings of the present invention.

What is claimed is:

1. A system for draining the intraocular cavity of an eye comprising:
a drainage shunt comprising:
an elongate inlet tube having a lumen, a first end, and a second end, wherein the first end of the elongate tube is inserted into an eye to place the lumen in contact with aqueous humor; and
a reservoir attached to the second end of the inlet tube, the reservoir being in fluid communication with the inlet tube lumen to receive fluid aqueous humor from the inlet tube, wherein the reservoir is attached to an eye; and
an inlet tube cover formed from a flexible material, the inlet tube cover having a length, a width, and a thickness, wherein the inlet tube cover comprises:
a channel formed along the length of the inlet tube cover on a bottom surface thereof;
wherein a section of the inlet tube between a point where the inlet tube is inserted into the eye and the reservoir is disposed in the channel; and
wherein the inlet tube cover is attached to the eye so that the inlet tube is held between the eye and the inlet tube cover.

2. The system of claim 1, wherein the inlet tube cover has a lower surface which is generally spherical and concave.

3. The system of claim 1, wherein the inlet tube cover has side edges which are curved and concave such that the inlet tube cover is generally hourglass shaped when viewed from above.

4. The system of claim 3, wherein the front edge of the inlet tube cover is narrower than a rear edge of the inlet tube cover.

5. The system of claim 3, wherein a front edge of the inlet tube cover is concave and curves inwardly from inlet tube cover front corners.

6. The system of claim 5, wherein a rear edge of the inlet tube cover is convex and curves outwardly from inlet tube cover rear corners.

7. The system of claim 1, wherein a lower surface of the inlet tube cover is textured.

8. The system of claim 1, wherein the inlet tube cover includes two wings formed on front corners of the inlet tube cover which extend outwardly from a center of the inlet tube cover.

9. The system of claim 1, wherein the inlet tube cover has two attachment points formed in a top surface thereof, and wherein the attachment points are depressions in the inlet tube cover which do not penetrate the inlet tube cover.

10. The system of claim 9, wherein the inlet tube cover has two opposed concave side edges which curve inwardly from front and rear corners and wherein the two attachment points are located adjacent a minimum width between the two opposed side edges.

11. The system of claim 1, wherein the reservoir comprises a valve which is attached to the second end of the inlet tube in fluid communication with the inlet tube lumen, and wherein the valve opens at a predetermined pressure to allow flow through the lumen of the inlet tube into the reservoir.

12. A system for draining the intraocular cavity of an eye comprising:
a drainage shunt comprising:
an elongate inlet tube having a lumen, a first end, and a second end, wherein the first end of the elongate tube is inserted into an eye to place the lumen in contact with aqueous humor; and
a reservoir attached to the second end of the inlet tube, the reservoir being in fluid communication with the inlet tube lumen to receive fluid aqueous humor from the inlet tube, wherein the reservoir is attached to an eye; and
an inlet tube cover formed from a flexible material, the inlet tube cover having a length, a width, and a thickness;
wherein the inlet tube cover has two opposed concave side edges which curve inwardly from front and rear corners such that the inlet tube cover is generally hourglass shaped when viewed from above; and
wherein the inlet tube cover is attached to the eye so that the inlet tube is held between the eye and the inlet tube cover.

13. The system of claim 12, wherein the inlet tube cover includes two wings formed on front corners of the inlet tube cover which extend outwardly from a center of the inlet tube cover.

14. The system of claim 12, wherein the front edge of the inlet tube cover is narrower than a rear edge of the inlet tube cover.

15. The system of claim 12, wherein a front edge of the inlet tube cover is concave and curves inwardly from inlet tube cover front corners.

16. The system of claim 12, wherein the inlet tube cover comprises a channel formed along the length of the inlet tube cover on a bottom surface thereof, and wherein a section of the inlet tube between a point where the inlet tube is inserted into the eye and the reservoir is disposed in the channel.

17. The system of claim 12, wherein the inlet tube cover has a lower surface which is generally spherical and concave.

18. The system of claim 12, wherein the inlet tube cover has two attachment points formed in a top surface thereof, and wherein the attachment points are depressions in the inlet tube cover which do not penetrate the inlet tube cover.

19. The system of claim 18, wherein the attachment points are located adjacent a minimum width between the two opposed side edges.

20. The system of claim 12, wherein the reservoir comprises a valve which is attached to the second end of the inlet tube in fluid communication with the inlet tube lumen, and wherein the valve opens at a predetermined pressure to allow flow through the lumen of the inlet tube into the reservoir.
